(19) United States
(12) Patent Application Publication
(10) Pub. No.: US 2026/0325857 A1
(43) Pub. Date: Oct. 22, 2026

(54) ADAPTIVE TRANSCUTANEOUS MAGNETIC STIMULATION THERAPY

(71) Applicant: The United States Government as represented by the Department of Veterans Affairs, Washington, DC (US)

(72) Inventor: Albert Y. Leung, Del Mar, CA (US)

(21) Appl. No.: 19/106,705

(22) PCT Filed: Aug. 30, 2023
(86) PCT No.: PCT/US2023/031503

§ 371 Date: Feb. 26, 2025

Related U.S. Application Data
(60) Provisional application No. 63/402,602, filed on Aug. 31, 2022.
(60) Provisional application No. 63/410,290, filed on Sep. 27, 2022.
(60) Provisional application No. 63/476,442, filed on Dec. 21, 2022.

(51) Int. Cl. A61N 2/00; A61N 2/02

(52) U.S. Cl. CPC A61N 2/006; A61N 2/008; A61N 2/02

(57) ABSTRACT

Adaptive magnetic stimulation therapy for a nerve injury including chronic peripheral nerve paid, e.g., through the use of a patient-machine interface (PMI).

### CROSS-REFERENCE TO RELATED APPLICATIONS

This application claims priority to U.S. Provisional Application No. 63/476,442, filed Dec. 21, 2022, U.S. Provisional Application No. 63/410,290, filed Sep. 27, 2022, and U.S. Provisional Application No. 63/402,602, filed Aug. 31, 2022. The entirety of each of these applications is incorporated into this application by reference.

### BACKGROUND

Post-traumatic neuropathic pain is a common occurrence in military and civilian populations due to non-surgical or surgical injuries. Nerve injury often results in the formation of neuroma or nerve entrapment, a condition leading to persistent neuropathic pain states with increases in ectopic activity at the site of injury or at the dorsal root ganglion (DRG) of the injured axons. Invasive measures such as surgical resection, nerve transposition, or local injection of steroid and local anesthetics at the neuroma site are often ineffective in alleviating neuropathic pain. The invasive nature of these interventions can also further exacerbate pain associated with pre-existing hypersensitive neuropathic pain states. Consequently, there is a need in the field of pain management for non-invasive therapy useful in treating post-traumatic neuropathic pain states.

### SUMMARY

This application describes a non-invasive neuromodulation modality, referred to as transcutaneous magnetic stimulation (tMS), to effectively and adaptively treat a nerve injury or neuropathic pain in a subject, including post-traumatic peripheral nerve pain and nerve pain at the site of a nerve injury.

In some embodiments, as illustrated in the general flow chart shown in FIG. 1, the treatment method (100) involves determining a starting magnetic stimulation intensity (102), defined as a percentage of a maximum dB/dt value achievable by a magnetic stimulation device. In some embodiments, determining the starting magnetic stimulation intensity (102) can be performed by a computing device (e.g., patient-machine interface (“PMI”) (112)) which is coupled to the magnetic stimulation device. After determining the starting intensity, the method can involve adjusting the stimulation intensity (104), e.g., via PMI (112), based on the subject's feedback to ultimately determine the subject's magnetic stimulation sensing threshold intensity (106). The subject's magnetic stimulation sensing threshold can then be adjusted based again on the subject's feedback to determine an appropriate treatment stimulation intensity (108). The subject's pain can then be treated at the treatment stimulation intensity (110), e.g., via PMI (112) which can optionally be adjusted according to a comfort rating provided by the subject during treatment. In some embodiments, the subject's feedback can be received by a computing device such as PMI (112) and the intensity of the applied magnetic field can be controlled by the computing device based on the subject's feedback and a predetermined treatment intensity paradigm. In some embodiments, the treatment method may be a closed-loop method.

Also described are systems that include a magnetic stimulation device and a computing device for performing the described treatment methods. A computing apparatus comprising a processor for performing the treatment method steps is also described. The computing apparatus for example may include one or more processors and computer-executable instructions, that when executed by the one or more processors, cause the apparatus to perform various or all steps of the described treatment method.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1 is a flowchart for an exemplary embodiment of the described adaptive tMS method.

FIG. 2A is a chart showing an exemplary protocol for determining the starting stimulation intensity based on a maximum dB/dt value achievable by the magnetic stimulation device.

FIG. 2B is a chart showing an exemplary sensing threshold determination paradigm.

FIG. 2C is a chart showing an exemplary treatment intensity determination paradigm.

FIG. 2D is a chart showing an exemplary final treatment intensity comfort level rating after determining the treatment intensity.

FIG. 3 is a photograph of an exemplary magnetic stimulation device as it is being used on a target area of a patient.

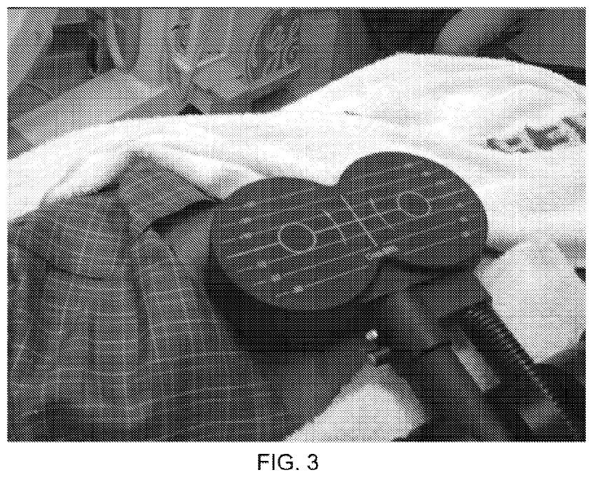

FIG. 4 is a block diagram depicting an exemplary computer-implemented environment capable of performing the described treatment method.

FIG. 5 is a block diagram depicting an exemplary computer-implemented environment capable of performing the described treatment method, which includes a remote computing device for receiving or providing input or data from or to a subject or clinician.

### DETAILED DESCRIPTION

In general, tMS offers an innovative, non-invasive, and non-contact means of neuromodulation in managing neuropathic pain via the use of a dynamic magnetic flux, which can affect neuronal functions by inducing localized neuronal depolarization. The method is similar to the use of transcranial magnetic stimulation (TMS), which uses electromagnetic principles to produce small electrical currents in neurons without skin contact or anesthetics. The method of pain neuromodulation without skin contact provides a major advantage in treating patients with tactile allodynia (painful sensitivity to non-painful stimuli).

When a current is passed through a magnetic stimulation device, such as a coil for example, a dynamic magnetic flux will pass through the skin and into the first few centimeters of the skin without attenuation. In some embodiments, a figure-of-eight coil can be used because it gives a focused dynamic magnetic flux stimulation from the center of the coil to the target site which can be marked with an extended cross-hair for alignment. This dynamic magnetic flux induced neuronal stimulation is more focused than other available direct current-based electrotherapies.

The inventor determined through a mechanistic nerve conduction study that the tMS treatment method may potentially restore lost nerve functions related to pain control. However, the inventor also discovered that treatment at a fixed dosing intensity can only generate short-term pain relief for neuropathic pain, without a more sustainable benefit. The inventor therefore devised a patient-specific, treatment-by-treatment intensity method which is more suitable for achieving a long-term sustainable benefit for patients suffering from chronic neuropathic pain. The patient-specific method involves a closed-loop form of treatment, which can optionally include pretreatment patient feedback, ongoing patient feedback, treatment intensity control, and further optimization as treatment progresses. In some embodiments, treatment settings can be adjusted automatically based on the subject's feedback in a closed-loop formed between the subject and the computing device or controller (e.g., without the need for input from a clinician). In some optional embodiments, this feedback from the subject can be provided entirely before the actual treatment of the subject begins. That is, in these optional embodiments, the subject can provide pretreatment, closed-loop feedback that is used to determine the optimal treatment settings, and then the optimal treatment settings can be applied once treatment begins. Optionally, in these aspects, it is contemplated that the treatment can be performed without further feedback from the subject.

A variety of nerve injuries can be treated. “Nerve injury” as used in this application refers to any injury resulting from physical, chemical, or disease-induced trauma to a peripheral nerve, which can lead for instance to structural or functional changes or restructuring of the nerve at the axonal or ganglion (cell body) levels. In some subjects, the nerve injury will result in peripheral nerve pain (the site of treatment) which can be chronic peripheral nerve pain.

### Treatment Method

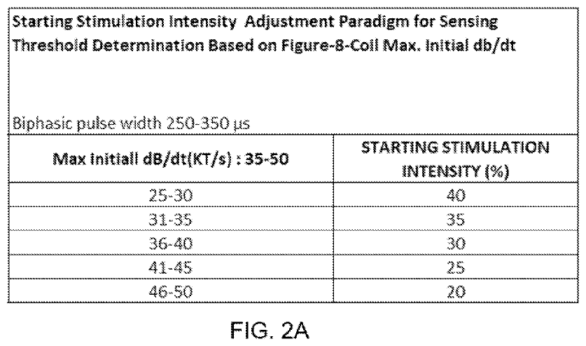

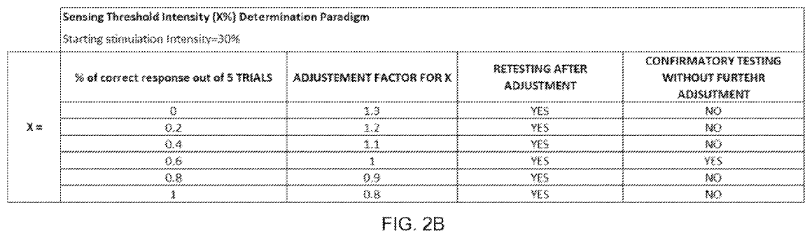

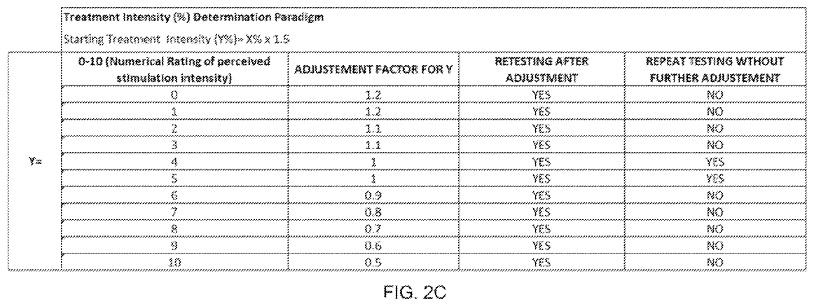

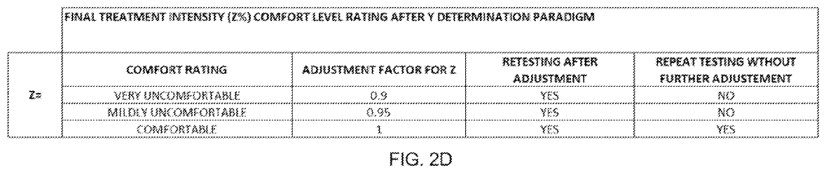

An exemplary embodiment of the treatment method is shown FIGS. 2A-D. As shown in FIG. 2A, the first step of the method involves determining a starting stimulation intensity. In general, the starting stimulation intensity can be based on the strength of the magnetic stimulation device. Magnetic stimulation devices approved for medical use have a corresponding magnetic field strength, which can be measured for example as a maximum dB/dt value, reported as T/s or KT/s. “dB/dt” refers to the ratio between the amount of change in amplitude of a magnetic field (dB) and the time it takes to make that change (dt). The maximum dB/dt value, as one skilled in the art will appreciate, will vary from device to device.

The chart in FIG. 2A provides an exemplary guide for determining the starting stimulation intensity as a percentage of the maximum dB/dt value of the magnetic stimulation device. As shown in the chart, the higher the maximum dB/dt value of the magnetic stimulation device, the lower the starting stimulation intensity. For example, for a magnetic stimulation device with a maximum dB/dt value between 36-40 KT/s, the starting simulation intensity can be 30% of the maximum dB/dt value, or 10.8-12 KT/s. The numbers will vary from device to device and subject to subject, with the goal of the first step being to determine a subject's sensing threshold intensity in a manner that is reasonably comfortable for the subject. For example, the starting stimulation intensity as a percentage of the maximum can be 40% for a device capable of producing a maximum initial dB/dt (KT/s) ranging from 25-30, 35% for a device capable of producing a maximum initial dB/dt (KT/s) ranging from 31-35, 30% for a device capable of producing a maximum initial dB/dt (KT/s) ranging from 36-40, 25% for a device capable of producing a maximum initial dB/dt (KT/s) ranging from 41-45, or 20% for a device capable of producing a maximum initial dB/dt (KT/s) ranging from 46-50.

At a given starting stimulation intensity, the subject's magnetic stimulation sensing threshold intensity can then be determined. A low frequency magnetic field can be delivered to the target area of the subject at the predetermined starting stimulation intensity, determined as a percentage of the maximum dB/dt value achievable by the magnetic stimulation device. In one embodiment, delivering the magnetic field to the target area of the subject comprises placing the magnetic stimulation device near the target area but not directly on the skin of the target area (e.g., from 0.5-2 cm or about 1 cm away from the target area).

In one embodiment of the method, the subject can indicate whether or not stimulation is felt at the target area at the starting stimulation intensity, and this response can be received at a computing device coupled to the magnetic stimulation device, either from the subject's input, gesture, or other response directly, or through a medical practitioner who is recording the subject's feedback. This step can be repeated over a plurality of trials, where an indication from the subject that stimulation is felt is a positive response and an indication that stimulation is not felt is a negative response.

FIG. 2B shows a chart of an exemplary paradigm for determining the subject's magnetic stimulation sensing threshold intensity, or in other words the magnetic field intensity at which the subject accurately perceives magnetic stimulation at the target area. As shown in FIG. 2B, in an exemplary five-trial paradigm, with a starting stimulation intensity of 30% of the maximum dB/dt value of the magnetic stimulation device, the computing device coupled to the magnetic stimulation device can receive the number of positive (correct) and negative (incorrect) responses from the subject at the starting stimulation intensity over the series of five trials. If for example after five trials the subject has indicated that stimulation was not felt five consecutive times, then the starting stimulation intensity can be adjusted by a factor of 1.3, and the process can be repeated again to arrive at the subject's proper stimulation sensing threshold intensity.

In the exemplary paradigm shown in FIG. 2B, the starting stimulation can be adjusted up or down by the computing device coupled to the magnetic stimulation device, based on the subject's positive or negative responses, until the subject indicates a positive (or correct) response 60% of the time, or three out of five trials. In some embodiments, the subject can be exposed to control trials, in which no magnetic stimulation is delivered to the target area, to determine whether or not the subject indicates a correct, negative response. If in the example in which the subject does not feel stimulation in any of the five trials, despite magnetic stimulation being delivered, the process can be repeated at 1.3 times the starting stimulation intensity. If the subject still does not feel stimulation in any of the five trials, the intensity can then be adjusted by another factor of 1.3 until the subject eventually reports 60% positive responses when stimulation intensity is applied. As is also shown in FIG. 2B, in some embodiments, even when the subject does report 60% positive responses with stimulation intensity applied, one or more optional retests or confirmation tests can be performed to ensure that the subject's responses are reproducible.

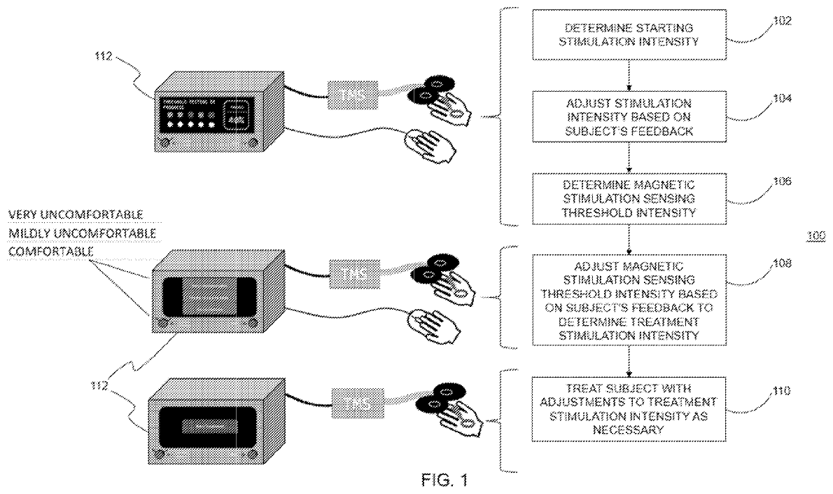

In one embodiment, determining the magnetic stimulation intensity can be done with the use of a computing device such as the PMI (112) depicted in FIG. 1. Specifically, once the patient is properly positioned, the sensing threshold can be determined via an Automatic Neurofeedback Sensing Threshold Determination System provided by the PMI (112), as further discussed herein. A magnetic stimulation device can be placed over the target area at a specified distance without touching the skin. Patients can provide feedback via a mouse or a touch screen interface linked to or provided as a component of the PMI (112). The subject can click the mouse (or provide other suitable input to the PMI) within a set time frame (e.g., 5 seconds) when the stimulation is felt. A correct response within the set time frame can be indicated by a green light (or other visible or audible indicator), whereas a missed response can be shown as a red light (or other visible or audible indicator that is distinguishable from the indicator associated with the correct response). Multiple responses within a set time frame can be indicated as a yellow light (or other visible or audible indicator that is distinguishable from the indicators associated with the correct and missed responses). The lowest stimulation amplitude at which a patient responds correctly at a preset number of successful trial thresholds can be automatically recorded and displayed on the PMI module screen as the tMS sensing threshold.

In one embodiment, an algorithm for determining the magnetic stimulation sensing threshold can include a method for decelerating the magnetic intensity as it changes directions from positive to negative. In this embodiment, the PMI for instance can start the magnetic stimulation intensity at zero and then increase at a fast rate until a certain number of correct responses are received by the subject, then the magnetic stimulation device can decrease intensity at a slower rate until a certain number of correct responses are received, and then for example increase at an even slower rate.

In various embodiments, the subject can provide feedback to the PMI or computing device through various modes, for example a push-button device, a remote, a mobile device application, a mouse, and the like, and any step of the treatment method.

After carrying out the exemplary magnetic stimulation sensing threshold intensity determination paradigm shown in FIG. 2B, the result is a determination of the magnetic stimulation intensity at which the subject accurately perceives a physical response at the target area, i.e., in units of T/s or KT/s. In some embodiments, this can be accomplished by the Automatic Neurofeedback Sensing Threshold Determination System. The next step in the exemplary process is a determination of a treatment stimulation intensity, or an intensity at which the subject's nerve injury will be treated.

Referring to the exemplary paradigm shown in FIG. 2C, determining the treatment stimulation intensity can involve adjusting the magnetic stimulation based on a numerical, perceived stimulation intensity rating provided by the subject and received by the computing device. Thus, a low frequency magnetic field can be delivered to the target area of the subject at the magnetic stimulation sensing threshold intensity or a minor adjusted variant thereof (e.g., 1.5 times the magnetic stimulation sensing threshold intensity as shown in FIG. 2C), and the subject can indicate a rating reflective of the intensity of stimulation.

In the example shown in FIG. 2C, the applied intensity can be adjusted upward or downward by an adjustment factor based on the subject's numerical rating of the perceived stimulation intensity. For example, the subject can rate the intensity on a 0-10 scale, with 0 being the subject's lowest perceived intensity rating and 10 being the highest. If the subject rates the intensity 0, the intensity can be adjusted by the computing device by an appropriate factor, e.g., 1.2, and the subject's perceived intensity can be re-evaluated. In general, this process can be repeated until the subject indicates a defined rating of perceived stimulation intensity (e.g., 4-5 in the example shown in FIG. 2C). Once the subject arrives at the pre-defined rating, optional retesting can be carried out to ensure the subject's responses are reproducible. The result from the exemplary paradigm depicted in FIG. 2C is the treatment stimulation intensity.

In one embodiment, the PMI (112) shown in FIG. 1 can be programmed to automatically instruct a magnetic stimulation device to deliver an initial treatment pulse at an intensity based on the PMI programmed algorithm (i.e., through an Automatic Neurofeedback Sensing Threshold Determination System). The PMI (112) can then issue a voice command (and/or display a visual (e.g., text-based) command) asking the patient to provide a rating of the treatment intensity by moving an indicator of the visual analogue scale displaced on a liquid-crystal display (LCD) monitor. If a response is not obtained with 10 seconds, the PMI (112) can issue another command asking the treatment recipient to rate the stimulation intensity. If a response is not obtained within 10 seconds after the stimulation delivery, then the PMI (112) can ask the patient if the testing process should be terminated. If the treatment recipient selects a YES response, the PMI (112) will stop the process. If the recipient selects the NO response, then the testing process will start again. Each time the PMI (112) registers the rating within 10 seconds after the stimulation, the PMI can automatically adjust the treatment intensity and re-run the test algorithm until the rating is within the predetermined ideal treatment feedback range. Once the treatment intensity has reached the ideal treatment intensity range, a green light (or other visual or audible indicator) can be activated (e.g., displayed on an LCD monitor). Additional fine-tuning of the treatment intensity can be conducted until the PMI LCD monitor displays three green lights (or provides another visual or audible indicator corresponding to predefined tuning requirements and/or thresholds).

The next phase of the method is the treatment phase. Referring to the exemplary paradigm shown in FIG. 2D, a low frequency magnetic field can be delivered to the target area of the subject to treat the subject's nerve injury. Optionally, a paradigm shown in FIG. 2D can then be employed, in which the subject provides feedback on the comfort level of the treatment stimulation intensity (e.g., very or mildly uncomfortable or comfortable), and the treatment intensity can be adjusted accordingly (e.g., by a factor of 0.9, 0.95, or 1, respectively). Additionally, optional retesting and repeat testing can be performed to ensure that the treatment stimulation intensity continues to be comfortable for the subject.

Referring again to FIG. 1, the treatment phase can in some embodiments be carried out with the use of PMI (112), e.g., optionally with the programmed Automatic Neurofeedback Sensing Threshold Determination System. PMI (112) can provide comfort level options, which can be selected by the patient upon delivery of a magnetic field. For example, if a subject's response is “VERY/MILDLY UNCOMFORTABLE,” then the PMI (112) can automatically adjust the output setting based on the programmed algorithm, deliver another magnetic pulse, and ask the subject by voice command (and/or visual (e.g., text-based) command) to rate the comfort level again. The process can be programmed to repeat until the “COMFORTABLE” choice is selected. The “START TREATMENT” tag can then appear on the PMI LCD/touch screen, prompting the provider to initiate treatment. Based on the determined treatment intensity, the PMI (112) can instruct the magnetic stimulation device to deliver treatment with a specific number of pulses and frequency. In some embodiments, the PMI (112) can adjust the treatment intensity at each subsequent session using an automated close-loop algorithm as described above. In various embodiments, the treatment method can include an initial five consecutive sessions of treatment delivered 1-3 days apart over a two-week period via the automated PMI (112) which is coupled to the magnetic stimulation device. The treatment method can in some embodiments include additional maintenance sessions delivered weekly for two months, then biweekly to monthly thereafter.

In some embodiments, the treatment algorithm can include an optional close-loop neurophysiological monitoring step for optimizing treatment. Specifically, enhanced beta-afferent sensory input can be associated with an analgesic benefit of magnetic stimulation. Thus, additional electrophysiological data including conduction velocity, onset/peak latency and peak amplitude can monitored and processed data can be incorporated in the feedback loop algorithm to provide additional automated treatment instruction to target specific neurophysiological parameters including but not limited to conduction velocity, onset/peak latency and peak amplitude for the compatible magnetic stimulation device.

Using the tMS adaptive treatment method, a variety of pain management therapies can be developed for long-term pain management. In some embodiments, a subject can be treated at the treatment stimulation intensity or a variant thereof based on the subject's perceived stimulation intensity feedback, every one to every three days during an initial two-week induction period. In one embodiment, after the initial two-week induction period, the treatment step (and optionally the comfort rating step) can be repeated every week for two months during a first maintenance period. In a further embodiment, after the first maintenance period, the treatment step (and optionally the comfort rating step) can be repeated every two weeks to every month during a second maintenance period. The treatment protocol can begin with feedback from the patient prior to treatment optimization.

### Magnetic Stimulation Devices

A variety of magnetic stimulation devices are compatible with the adaptive tMS method. In one embodiment, the magnetic stimulation device is a transcutaneous magnetic stimulator (tMS). In a further embodiment, the tMS stimulator comprises a rotatable magnetic coil disposed within a positionable housing. The coil can include a left coil and a right coil, and the left coil and the right coil can rotate with respect to one another to adjust a focal point of magnetic stimulation being delivered to the target area of the subject.

In a further embodiment, the left coil and the right coil rotate in a range from a first approximately planar configuration in which an angle between the left coil and the right coil is approximately 180 degrees to a second rotated configuration in which the angle between each coil is an obtuse angle. In another embodiment, the left coil and the right coil rotate up to 30 degrees from the first approximately planar configuration to the second rotated configuration.

In some embodiments, the tMS stimulator comprises an accelerometer configured to detect a magnitude and direction of movement of the tMS stimulator. In another embodiment, the tMS stimulator includes a proximity sensor configured to determine whether the tMS stimulator is at the target body area. In one embodiment, the left coil and the right coil rotate simultaneously via a central gear mechanism. Optionally, in one embodiment, the tMS stimulator is configured to produce an output of up to 3 Tesla with between 20-65 kTesla/second of instantaneous flux at a frequency of approximately 0.2 Hertz (Hz) to approximately 5 Hz. Figure-eight style tMS devices including those having the features described above, and their general use, are described in U.S. Pat. Nos. 10,369,373 and 11,273,317, both of which are incorporated into this application by reference in their entirety, for their teachings of tMS devices and their use. Other exemplary magnetic stimulation devices include those available commercially from MAGVENTURE and MAGSTIM. In some optional embodiments, the tMS device is programmed or otherwise configured either through a computing device, control module, or PMI such as the one depicted in FIG. 1, to produce an output that is outside the range of up to 3 Tesla with 20 kTesla/sec. of instantaneous flux and/or outside the frequency range of about 0.2 Hertz (Hz) to about 5 Hz. Thus, for example, the tMS device can be configured to operate at a frequency below about 0.2 Hertz and/or above about 5 Hz. Similarly, in some optional embodiments, the tMS device can be configured to produce an output that is above 3 Tesla, and/or at an instantaneous flux of less than about 20 kTesla/sec. (e.g., at 15 kTesla/sec. or less) or more than about 20 kTesla/sec. (e.g., at 25 kTesla/sec. or more). In one specific embodiment, the tMS device is incapable of producing an output of up to 3 Tesla with 20 kTesla/sec. of instantaneous flux at a frequency of approximately 0.2-5 Hz.

### Computing Devices

Any suitable computing device can be used in conjunction with the treatment method. In general, the computing device can be coupled (e.g., communicatively coupled) to the magnetic stimulation device and configured to adjust the stimulation intensity according to the treatment method paradigms, in response to feedback from the subject received at the computing device. The subject's feedback can be manually input into the computing device, e.g., by the subject or a medical practitioner through a touchscreen, keyboard, or alternatively can be sensed by the computing device based on a physical or verbal response from the subject or a medical practitioner.

Examples of suitable computing devices include mobile or stationary computing devices that can include a variety of hardware and software components (e.g., handheld computers, desktop computers, laptops, notebooks, tablets, and the like). The computing device can be in wireless or wired communication with other computing devices, a wireless network, or a cellular or satellite network. In general, the computing device can include a controller or processor (e.g., a signal processor, an image processing unit, a microprocessor, or other control and processing logic circuitry or software). The controller or processor can perform tasks such as signal encoding, image processing, data processing, input/output processing, power control, and other functions, including integration with and control of the magnetic stimulation device. The computing device can include an operating system for the support of one or more applications, e.g., medical software applications among others.

The mobile computing device can include memory. The memory can include a non-removable memory, removable memory, or both. Non-removable memory (or embedded memory) may include random access memory (RAM), read only memory (ROM), flash memory, hard disk drives, or other known memory storage. Removable memory can include a flash memory card (for example SD (Secure Digital) cards, memory sticks, Subscriber Identity Modules (SIM) cards, or other memory storage technologies such as smart cards. The memory can be used to store data and computer executable instructions for executing the operating system and the application(s) on the device. Exemplary data can include text, images, sound files, video material, or other data.

The computing device can support one or more input devices, such as a touch screen, a microphone, a camera, an integrated or external keyboard, a trackball, and one or more output devices, such as a speaker or display. Other possible output devices can include piezoelectric or other tactile output devices. Any of the input devices and output devices can be internal, external, or removably attached to the computing device. External input and output devices can communicate with the computing device via a wired or wireless connection. In some embodiments, a touch screen and display can be combined into a single input/output device.

The computing device can provide one or more natural user interfaces. For example, an operating system or application can include language recognition software as part of the voice user interface, while allowing the user to operate the device via voice commands. Moreover, the computing device can include input devices and software that allow a user to interact with the device via a user's spatial gesture. For example, a user's spatial gesture can be detected and interpreted to provide input in response to an applied magnetic stimulation intensity. In addition, the device can recognize gestures made by the device itself, such as detecting a tap gesture.

Referring to FIG. 1, one exemplary computing device can include the PMI (112) which is coupled to the magnetic stimulation device. The PMI can be a standalone medical device which meets applicable regulatory and safety requirement and enables the delivery of an automated close-loop treatment with any compatible TMS machine. In some embodiments, software controlling the circuit board can be programmed with an open source platform such as Python. In one embodiment, the device can have a dimension of about 10″ W×6″ H×5″ D and can contain an LCD screen for displaying testing response and intensity output. In some embodiments, the PMI can comprise 1-2 USB port(s) for a mouse and the TMS connection in addition to a serial port for connecting to a TMS machine. In some embodiments, the PMI can include a touch screen control option which can be applied for both obtaining feedback and inputting treatment initiation “Start” or “Reset” commands. Existing external control software such as MagPy, RAPID2, or Magic Toolbox can allow ad hoc control of a magnetic stimulation device.

As will be appreciated by one skilled in the art, hardware, software, or a combination of software and hardware may be used with the disclosed treatment method. Furthermore, a computer program product on a computer-readable storage medium (e.g., non-transitory) having processor-executable instructions (e.g., computer software) can be included in the computer-readable storage medium. Any suitable computer-readable storage medium may be utilized including hard disks, CD-ROMs, optical storage devices, magnetic storage devices, memristors, Non-Volatile Random Access Memory (NVRAM), flash memory, or a combination thereof.

The various treatment method steps may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

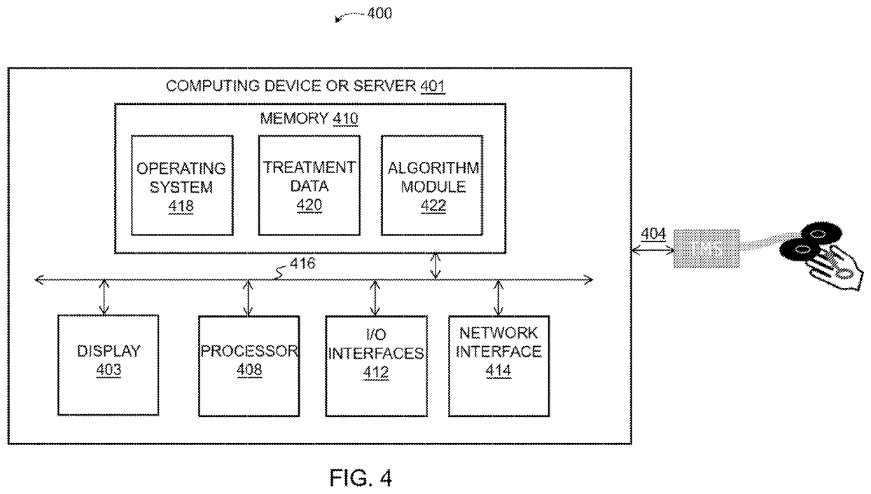

FIG. 4 shows a block diagram depicting an environment 400 comprising non-limiting examples of a computing device or server 401 and TMS stimulation device connected through a connection 404 (which in some embodiments may be any wired or wireless connection or network). The computing device or server 401 can comprise one or multiple computers configured to store treatment algorithms 422, treatment data 420, and the like. In exemplary aspects, it is contemplated that the PMI 112 can be or comprise a computing device 401 as disclosed and described herein.

The computing device or server 401 can be a digital computer that, in terms of hardware architecture, generally includes a display 403, a processor 408, a memory system 410, input/output (I/O) interfaces 412, and in some embodiments, network interfaces 414. These components can be communicatively coupled via a local interface 416, for example. The local interface 416 can be, for example, one or more buses or other wired or wireless connections, as is known in the art. The local interface 416 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 408 can be a hardware device for executing software, particularly that stored in memory system 410. The processor 408 can be any custom made or commercially available processor, a central processing unit (CPU), an auxiliary processor among several processors associated with the computing device or server 401, a semiconductor-based microprocessor (in the form of a microchip or chip set), or generally any device for executing software instructions. When the computing device or server 401 is in operation, the processor 408 can be configured to execute software stored within the memory system 410, to communicate data to and from the memory system 410, and to generally control operations of the computing device or server 401 pursuant to the software.

The I/O interfaces 412 can be used to receive user input from, and/or for providing system output to, one or more devices or components, including for example the TMS stimulation device. Subject or clinician input can be provided via, for example, a keyboard and/or a mouse, or in some embodiments a display 403 configured to receive input in a touch-screen format. System output can be provided via the display 403 and in some embodiments, a printer (not shown), for instance when it is desirable to keep hard copies of patient files. I/O interfaces 412 can include, for example, a serial port, a parallel port, a Small Computer System Interface (SCSI), an infrared (IR) interface, a radio frequency (RF) interface, and/or a universal serial bus (USB) interface.

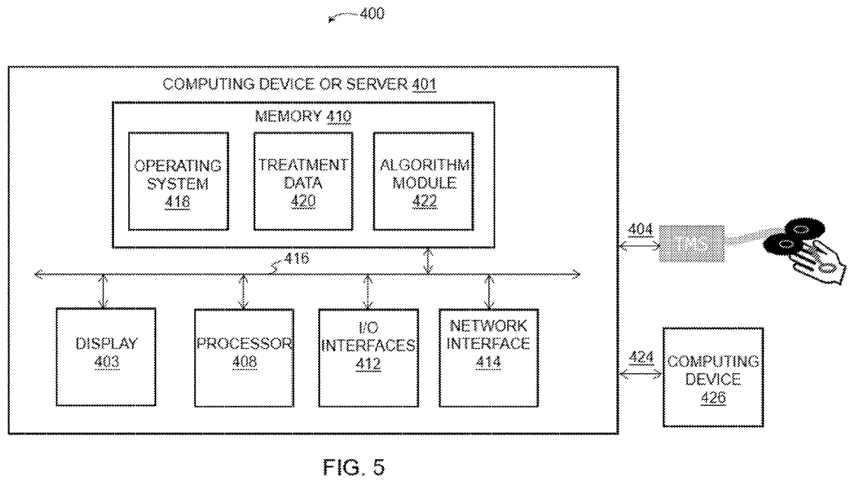

In a further embodiment depicted in FIG. 5, the computing device or server 401 can be communicatively coupled to a remote computing device 426 through wired or wireless connection 424. The remote computing device 426 can be any of those devices described above, for example, a tablet, a phone, computer, or any computing device capable of receiving input from a subject or clinician as related to the described treatment method. The computing device 426 can include for example a keyboard (external or internal), touchpad, mouse, and the like, which can enable input from the subject or clinician. Thus, in use, rather than providing inputs directly into the computing device 401, it is contemplated that the subject or clinician can provide inputs to the remote computing device 426, which in turn can be in communication with the computing device 401 through connection 424 (and the I/O interfaces 412).

Referring back to FIG. 4, in an environment which is not entirely wired, the network interface 414 for example can be used to transmit and receive data from the computing device or server 401 on the network 404. The network interface 414 may include, for example, a 8BaseT Ethernet Adaptor, a 80BaseT Ethernet Adaptor, a LAN PHY Ethernet Adaptor, a Token Ring Adaptor, a wireless network adapter (e.g., WiFi, cellular, satellite), or any other suitable network interface device. The network interface 414 may include address, control, and/or data connections to enable appropriate communications on the network 404.

The memory system 410 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 410 may incorporate electronic, magnetic, optical, and/or other types of storage media. The memory system 410 can have a distributed architecture, where various components are situated remote from one another, but can be accessed by the processor 408.

The software in memory system 410 may include one or more software programs, each of which comprises an ordered listing of executable instructions for implementing logical functions. In the example of FIG. 4, the software in the memory system 410 of the computing device or server 401 can comprise the algorithm module 422 (or subcomponents thereof), treatment data 420, and a suitable operating system (O/S) 418. The operating system 418 controls the execution of other computer programs and provides scheduling, input-output control, file and data management, memory management, and communication control and related services.

For purposes of illustration, application programs and other executable program components such as the operating system 418 are illustrated as discrete blocks, although it is recognized that such programs and components can reside at various times in different storage components of the computing device or server 401. An implementation of the treatment algorithm module 422 can be stored on or transmitted across some form of computer readable media.

Any of the disclosed treatment methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

### Exemplary Embodiments

The following are exemplary embodiments to further illustrate the description, claims, and appended drawings.

(1) Embodiment 1: A closed-loop method of treating a nerve injury in a subject comprising:
a) delivering, through a magnetic stimulation device, a low frequency magnetic field at a starting stimulation intensity at a target area of the subject, the starting stimulation intensity being defined as a percentage of a maximum dB/dt value achievable by the magnetic stimulation device;
b) receiving, by a computing device coupled to the magnetic stimulation device, an indication from the subject that stimulation is felt or not felt at the target area during a plurality of trials, wherein an indication that stimulation is felt is a positive response and an indication that stimulation is not felt is a negative response;
c) adjusting, by the computing device, the starting stimulation intensity upward or downward to provide an adjusted stimulation intensity based on the subject's positive and negative responses from step b);
d) repeating step b) at the adjusted stimulation intensity until the subject indicates a defined number of positive responses, wherein the adjusted stimulation intensity associated with the defined number of positive responses corresponds to a magnetic stimulation sensing threshold intensity;
e) delivering, through the magnetic stimulation device, a low frequency magnetic field at the magnetic stimulation sensing threshold intensity or an upward adjustment thereof to the target area of the subject;
f) receiving, by the computing device, a rating from the subject reflective of an intensity of the low frequency magnetic field felt by the subject during step e);
g) adjusting, by the computing device, the magnetic stimulation sensing threshold intensity upward or downward to provide an adjusted magnetic stimulation sensing threshold intensity based on the subject's rating of the intensity of the low frequency magnetic field;
h) repeating steps e) and f) until the subject indicates a defined rating of perceived stimulation intensity, wherein the adjusted magnetic stimulation sensing threshold intensity associated with the defined rating of perceived stimulation intensity corresponds to a subject-specific treatment stimulation intensity;
i) delivering, through the magnetic stimulation device, a low frequency magnetic field at the treatment stimulation intensity, thereby treating the nerve injury in the subject.

(2) Embodiment 1, further comprising j) optionally receiving during step i), by the computing device, a comfort rating from the subject reflective of the intensity of the low frequency magnetic field felt by the subject during step i).

(3) Embodiment 2, further comprising optionally adjusting, by the computing device, the treatment stimulation intensity until the subject indicates a defined comfort rating.

(4) Any preceding Embodiment, wherein the starting stimulation intensity ranges from about 20% to about 40% of the maximum dB/dt value achievable by the magnetic stimulation device.

(5) Any preceding Embodiment, wherein during step c), the stimulation intensity is adjusted upward when the subject indicates less than 60% positive responses and downward when the subject indicates more than 60% positive responses.

(6) Any preceding Embodiment, wherein at least step i) is repeated every one to three days during an initial two-week induction period.

(7) Any preceding Embodiment, wherein after the initial two-week induction period, at least step i) is repeated every week for two months during a first maintenance period.

(8) Embodiment (5), wherein after the first maintenance period, at least step i) is repeated every two weeks to every month during a second maintenance period.

(9) Any preceding Embodiment, wherein the magnetic stimulation device is a transcutaneous magnetic stimulator (tMS).

(10) Embodiment (9), wherein the tMS stimulator comprises a rotatable magnetic coil disposed within a positionable housing; wherein the coil includes a left coil and a right coil, and wherein the left coil and the right coil rotate with respect to one another to adjust a focal point of magnetic stimulation being delivered to the target area of the subject.

(11) Embodiment (10), wherein the left coil and the right coil rotate in a range from a first approximately planar configuration in which an angle between the left coil and the right coil is approximately 180 degrees to a second rotated configuration in which the angle between each coil is an obtuse angle.

(12) Embodiment (10) or (11), wherein the left coil and the right coil rotate up to 30 degrees from the first approximately planar configuration to the second rotated configuration.

(13) Embodiment (10), (11), or (12), wherein the tMS stimulator further comprises an accelerometer configured to detect a magnitude and direction of movement of the tMS stimulator.

(14) Any one of Embodiments (10)-(13), wherein the tMS stimulator further includes a proximity sensor configured to determine whether the tMS stimulator is at the target body area.

(15) Any one of Embodiments (10)-(14), wherein the left coil and the right coil rotate simultaneously via a central gear mechanism.

(16) Any preceding Embodiment, wherein the computing device includes a patient-machine interface for (i) receiving input from the subject or a clinician and/or (ii) outputting information to the subject, the clinician, or the magnetic stimulation device.

(17) Embodiment 17: A system for treating a nerve injury, the system comprising:
a magnetic stimulation device; and
a computing device communicatively coupled to the magnetic stimulation device, wherein the computing device is configured to:
receive, following delivery by the magnetic stimulation device of a low frequency magnetic field at a starting stimulation intensity at a target area of a subject, an indication from the subject that stimulation is felt or not felt at the target area during a plurality of trials, wherein an indication that stimulation is felt is a positive response and an indication that stimulation is not felt is a negative response;
adjust the starting stimulation intensity upward or downward to provide an adjusted stimulation intensity based on the subject's positive and negative responses from the first step;
repeat the first step at the adjusted stimulation intensity until the subject indicates a defined number of positive responses, wherein the adjusted stimulation intensity associated with the defined number of positive responses corresponds to a magnetic stimulation sensing threshold intensity;
receive, following delivery by the magnetic stimulation device of a low frequency magnetic field at the magnetic stimulation sensing threshold intensity or an upward adjustment thereof to the target area of the subject, a rating from the subject reflective of an intensity of the low frequency magnetic field felt by the subject during the third step;
adjust the magnetic stimulation sensing threshold intensity upward or downward to provide an adjusted magnetic stimulation sensing threshold intensity based on the subject's rating of the intensity of the low frequency magnetic field; and repeat the fourth step until the subject indicates a defined rating of perceived stimulation intensity, wherein the adjusted magnetic stimulation sensing threshold intensity associated with the defined rating of perceived stimulation intensity corresponds to a subject-specific treatment stimulation intensity,
wherein the magnetic stimulation device is configured to deliver a low frequency magnetic field at the treatment stimulation intensity.

(18) The system of Embodiment (17), wherein, during delivery of the low frequency magnetic field at the treatment stimulation intensity to the subject, the computing device is configured to receive a comfort rating from the subject reflective of the intensity of the low frequency magnetic field felt by the subject.

(19) The system of Embodiment (17), wherein the computing device is configured to adjust the treatment stimulation intensity until the subject indicates a defined comfort rating.

(20) The system of Embodiment (18) or (19), wherein the computing device includes a patient-machine interface for (i) receiving input from the subject or a clinician and/or (ii) outputting information to the subject, the clinician, or the magnetic stimulation device.

(21) an Apparatus Comprising:
one or more processors; and
computer-executable instructions that, when executed by the one or more processors, cause the apparatus to:
   a) deliver, through a magnetic stimulation device communicatively coupled to the apparatus, a low frequency magnetic field at a starting stimulation intensity at a target area of a subject, the starting stimulation intensity being defined as a percentage of a maximum dB/dt value achievable by the magnetic stimulation device;
   b) receive an indication from the subject that stimulation is felt or not felt at the target area during a plurality of trials, wherein an indication that stimulation is felt is a positive response and an indication that stimulation is not felt is a negative response;
   c) adjust the starting stimulation intensity upward or downward to provide an adjusted stimulation intensity based on the subject's positive and negative responses from step b);
   d) repeat step b) at the adjusted stimulation intensity until the subject indicates a defined number of positive responses, wherein the adjusted stimulation intensity associated with the defined number of positive responses corresponds to a magnetic stimulation sensing threshold intensity;
   e) deliver, through the magnetic stimulation device, a low frequency magnetic field at the magnetic stimulation sensing threshold intensity or an upward adjustment thereof to the target area of the subject;
   f) receive a rating from the subject reflective of an intensity of the low frequency magnetic field felt by the subject during step e);
   g) adjust the magnetic stimulation sensing threshold intensity upward or downward to provide an adjusted magnetic stimulation sensing threshold intensity based on the subject's rating of the intensity of the low frequency magnetic field;
   h) repeat steps e) and f) until the subject indicates a defined rating of perceived stimulation intensity, wherein the adjusted magnetic stimulation sensing threshold intensity associated with the defined rating of perceived stimulation intensity corresponds to a treatment stimulation intensity;
   i) deliver, through the magnetic stimulation device, a low frequency magnetic field at the treatment stimulation intensity, thereby treating the nerve injury in the subject.

(22) The apparatus of Embodiment (21), wherein the computer-executable instructions further cause the apparatus to receive during step i) a comfort rating from the subject reflective of the intensity of the low frequency magnetic field felt by the subject during step i).

(23) The apparatus of Embodiment (22), wherein the computer-executable instructions further cause the apparatus to adjust the treatment stimulation intensity until the subject indicates a defined comfort rating.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other methods and systems for carrying out the several purposes of this disclosure. As a result, the claims should not be considered as limited by the description or examples.

What is claimed is:

1. A closed-loop method of treating a nerve injury in a subject comprising:
a) delivering, through a magnetic stimulation device, a low frequency magnetic field at a starting stimulation intensity at a target area of the subject, the starting stimulation intensity being defined as a percentage of a maximum dB/dt value achievable by the magnetic stimulation device;
b) receiving, by a computing device coupled to the magnetic stimulation device, an indication from the subject that stimulation is felt or not felt at the target area during a plurality of trials, wherein an indication that stimulation is felt is a positive response and an indication that stimulation is not felt is a negative response;
c) adjusting, by the computing device, the starting stimulation intensity upward or downward to provide an adjusted stimulation intensity based on the subject's positive and negative responses from step b);
d) repeating step b) at the adjusted stimulation intensity until the subject indicates a defined number of positive responses, wherein the adjusted stimulation intensity associated with the defined number of positive responses corresponds to a magnetic stimulation sensing threshold intensity;
e) delivering, through the magnetic stimulation device, a low frequency magnetic field at the magnetic stimulation sensing threshold intensity or an upward adjustment thereof to the target area of the subject;
f) receiving, by the computing device, a rating from the subject reflective of an intensity of the low frequency magnetic field felt by the subject during step e);
g) adjusting, by the computing device, the magnetic stimulation sensing threshold intensity upward or downward to provide an adjusted magnetic stimulation sensing threshold intensity based on the subject's rating of the intensity of the low frequency magnetic field;
h) repeating steps e) and f) until the subject indicates a defined rating of perceived stimulation intensity, wherein the adjusted magnetic stimulation sensing threshold intensity associated with the defined rating of perceived stimulation intensity corresponds to a subject-specific treatment stimulation intensity;
i) delivering, through the magnetic stimulation device, a low frequency magnetic field at the treatment stimulation intensity, thereby treating the nerve injury in the subject.

2. The method of claim 1, further comprising j) optionally receiving during step i), by the computing device, a comfort rating from the subject reflective of the intensity of the low frequency magnetic field felt by the subject during step i).

3. The method of claim 2, further comprising optionally adjusting, by the computing device, the treatment stimulation intensity until the subject indicates a defined comfort rating.

4. The method of claim 1, wherein the starting stimulation intensity ranges from about 20% to about 40% of the maximum dB/dt value achievable by the magnetic stimulation device.

5. The method of claim 1, wherein during step c), the stimulation intensity is adjusted upward when the subject indicates less than 60% positive responses and downward when the subject indicates more than 60% positive responses.

6. The method of claim 1, wherein at least step i) is repeated every one to three days during an initial two-week induction period.

7. The method of claim 2, wherein after the initial two-week induction period, at least step i) is repeated every week for two months during a first maintenance period.

8. The method of claim 7, wherein after the first maintenance period, at least step i) is repeated every two weeks to every month during a second maintenance period.

9. The method of claim 1, wherein the magnetic stimulation device is a transcutaneous magnetic stimulator (tMS).

10. The method of claim 9, wherein the tMS stimulator comprises a rotatable magnetic coil disposed within a positionable housing; wherein the coil includes a left coil and a right coil, and wherein the left coil and the right coil rotate with respect to one another to adjust a focal point of magnetic stimulation being delivered to the target area of the subject.

11. The method of claim 10, wherein the left coil and the right coil rotate in a range from a first approximately planar configuration in which an angle between the left coil and the right coil is approximately 180 degrees to a second rotated configuration in which the angle between each coil is an obtuse angle.

12. The method of claim 10, wherein the left coil and the right coil rotate up to 30 degrees from the first approximately planar configuration to the second rotated configuration.

13. The method of claim 10, wherein the tMS stimulator further comprises an accelerometer configured to detect a magnitude and direction of movement of the tMS stimulator.

14. The method of claim 10, wherein the tMS stimulator further includes a proximity sensor configured to determine whether the tMS stimulator is at the target body area.

15. The method of claim 10, wherein the left coil and the right coil rotate simultaneously via a central gear mechanism.

16. The method of claim 1, wherein the computing device includes a patient-machine interface for (i) receiving input from the subject or a clinician and/or (ii) outputting information to the subject, the clinician, or the magnetic stimulation device.

17. A system for treating a nerve injury, the system comprising:
a magnetic stimulation device; and
a computing device communicatively coupled to the magnetic stimulation device, wherein the computing device is configured to:
(1) receive, following delivery by the magnetic stimulation device of a low frequency magnetic field at a starting stimulation intensity at a target area of a subject, an indication from the subject that stimulation is felt or not felt at the target area during a plurality of trials, wherein an indication that stimulation is felt is a positive response and an indication that stimulation is not felt is a negative response;
(2) adjust the starting stimulation intensity upward or downward to provide an adjusted stimulation intensity based on the subject's positive and negative responses from step (1);
(3) repeat step (1) at the adjusted stimulation intensity until the subject indicates a defined number of positive responses, wherein the adjusted stimulation intensity associated with the defined number of positive responses corresponds to a magnetic stimulation sensing threshold intensity;
(4) receive, following delivery by the magnetic stimulation device of a low frequency magnetic field at the magnetic stimulation sensing threshold intensity or an upward adjustment thereof to the target area of the subject, a rating from the subject reflective of an intensity of the low frequency magnetic field felt by the subject during step (3);
(5) adjust the magnetic stimulation sensing threshold intensity upward or downward to provide an adjusted magnetic stimulation sensing threshold intensity based on the subject's rating of the intensity of the low frequency magnetic field; and
(6) repeat step (4) until the subject indicates a defined rating of perceived stimulation intensity, wherein the adjusted magnetic stimulation sensing threshold intensity associated with the defined rating of perceived stimulation intensity corresponds to a subject-specific treatment stimulation intensity, wherein the magnetic stimulation device is configured to deliver a low frequency magnetic field at the treatment stimulation intensity.

18. The system of claim 17, wherein, during delivery of the low frequency magnetic field at the treatment stimulation intensity to the subject, the computing device is configured to receive a comfort rating from the subject reflective of the intensity of the low frequency magnetic field felt by the subject.

19. The system of claim 18, wherein the computing device is configured to adjust the treatment stimulation intensity until the subject indicates a defined comfort rating.

20. The method of claim 17, wherein the computing device includes a patient-machine interface for (i) receiving input from the subject or a clinician and/or (ii) outputting information to the subject, the clinician, or the magnetic stimulation device.

21. An apparatus comprising:
one or more processors; and
computer-executable instructions that, when executed by the one or more processors, cause the apparatus to:
a) deliver, through a magnetic stimulation device communicatively coupled to the apparatus, a low frequency magnetic field at a starting stimulation intensity at a target area of a subject, the starting stimulation intensity being defined as a percentage of a maximum dB/dt value achievable by the magnetic stimulation device;
b) receive an indication from the subject that stimulation is felt or not felt at the target area during a plurality of trials, wherein an indication that stimulation is felt is a positive response and an indication that stimulation is not felt is a negative response;
c) adjust the starting stimulation intensity upward or downward to provide an adjusted stimulation intensity based on the subject's positive and negative responses from step b);
d) repeat step b) at the adjusted stimulation intensity until the subject indicates a defined number of positive responses, wherein the adjusted stimulation intensity associated with the defined number of positive responses corresponds to a magnetic stimulation sensing threshold intensity;
e) deliver, through the magnetic stimulation device, a low frequency magnetic field at the magnetic stimulation sensing threshold intensity or an upward adjustment thereof to the target area of the subject;
f) receive a rating from the subject reflective of an intensity of the low frequency magnetic field felt by the subject during step e);
g) adjust the magnetic stimulation sensing threshold intensity upward or downward to provide an adjusted magnetic stimulation sensing threshold intensity based on the subject's rating of the intensity of the low frequency magnetic field;
h) repeat steps e) and f) until the subject indicates a defined rating of perceived stimulation intensity, wherein the adjusted magnetic stimulation sensing threshold intensity associated with the defined rating of perceived stimulation intensity corresponds to a treatment stimulation intensity;
i) deliver, through the magnetic stimulation device, a low frequency magnetic field at the treatment stimulation intensity, thereby treating the nerve injury in the subject.

22. The apparatus of claim 21, wherein the computer-executable instructions further cause the apparatus to receive during step i) a comfort rating from the subject reflective of the intensity of the low frequency magnetic field felt by the subject during step i).

23. The apparatus of claim 22, wherein the computer-executable instructions further cause the apparatus to adjust the treatment stimulation intensity until the subject indicates a defined comfort rating.
